(19) United States
(12) Patent Application Publication
(10) Pub. No.: US 2017/0132364 A1
(43) Pub. Date: May 11, 2017

(54) NON-INVASIVE PRENATAL TESTING METHOD BASED ON GENOME-WIDE NORMALIZED SCORE

(71) Applicant: WELGENE BIOTECH CO., LTD., Taipei City (TW)

(72) Inventors: Chen-Hsiang YEANG, Taipei City (TW); Ming CHEN, Taichung City (TW); Hung-Wei SHU, Taipei City (TW); Gwo-Chin MA, Taichung City (TW); Yi-Shin LIN, Taipei City (TW); Shun-Min CHANG, Taipei City (TW); I-Fen CHAN, Taipei City (TW); Shou-Jen KUO, Taipei City (TW)

(73) Assignee: WELGENE BIOTECH CO., LTD., Taipei City (TW)

(21) Appl. No.: 15/413,777

(22) Filed: Jan. 24, 2017

Related U.S. Application Data
(63) Continuation-in-part of application No. 14/184,988, filed on Feb. 20, 2014.

(30) Foreign Application Priority Data
Sep. 3, 2013 (TW) 102131625

(51) Int. Cl. G06F 19/22; C12Q 1/68; G06F 19/24

(52) U.S. Cl. CPC G06F 19/22; G06F 19/24; C12Q 1/6883

(57) ABSTRACT

Provided is a non-invasive prenatal testing method to test whether a target fetus has autosomal aneuploidy. The method includes: preparing control plasma samples and a target plasma sample; sequencing and obtaining the amount of total cfDNA and the amount of cfDNA of target chromosome k from each of the control plasma samples and the target plasma sample; respectively obtaining yₖ values from the control plasma samples and the target plasma sample, wherein each yₖ value is the ratio of the amount of cfDNA of target chromosome k to the amount of total cfDNA in each of the plasma samples; obtaining a mₖ value; obtaining a target Rₖ value, which is a normalized ratio of the target yₖ value to the mₖ value; and comparing whether the target Rₖ value is significantly different from the reference dataset.

### CROSS-REFERENCE TO RELATED PATENT APPLICATION

This application is a continuation-in-part application and claims priority from subject matter disclosed in the earlier filed patent application Ser. No. 14/184,988, filed on Feb. 20, 2014, which is incorporated herein in its entirety by reference.

### BACKGROUND OF THE INVENTION

1. Field of the Invention

The present invention relates to prenatal examination methods for calculating the copy number of fetal chromosomes, and more particularly, to a non-invasive test in which a pregnant woman's blood is drawn and analyzed to determine whether the fetus' chromosomes are aneuploidy.

2. Description of the Prior Arts

Prenatal diagnosis is performed on a fetus to detect chromosome aberrations and genetic disorders. Amniocentesis is the commonest invasive method for screening for fetal chromosomal abnormalities, such as aneuploidies which is a major cause of perinatal morbidity and mortality.

Amniocentesis, which is technically based on cell culture and deoxyribonucleic acid (DNA) analysis, is a medical procedure used in prenatal diagnosis, in which a needle is inserted through a pregnant woman's abdominal wall, then through the wall of the uterus, and finally into the amniotic sac surrounding a developing fetus, to allow a small amount of amniotic fluid, which contains fetal cells, to be sampled from the amniotic sac, and the fetal DNA is examined for genetic and congenital abnormalities. With the fetal DNA being examined directly, the precision of amniocentesis is beyond doubt. The best time to perform amniocentesis is 16^{th} to 18^{th} gestational weeks. Although amniocentesis is reliable and yields an absolutely accurate result, it is an invasive medical procedure and thus carries a 0.1% to 0.2% chance of miscarriage and a 0.05% chance of physical injury to the fetus. In view of the aforesaid drawbacks of the prior art, medical researchers are developing various methods for screening for chromosomal abnormalities.

In 1997, Professor Dennis Lo Yuk Ming, et al. discovered that cell-free fetal DNA (cffDNA) fragments could be extracted from a pregnant woman's plasma. The discovery enables non-invasive prenatal chromosomal test to take an innovative step forward, that is, drawing a pregnant woman's blood to analyze chromosomal aneuploidy by Next Generation Sequencing (NGS). However, the aforesaid innovative technique is still flawed with errors, because it does not obtain fetal chromosomes directly.

A conventional maternal blood screening method usually identifies a risky group by means of chromosome sequencing number expected value (Z-score) and performs evaluation by using the detection rate and the false positive rate as the screening criteria. The method is hereunder referred to as Z-score technique.

Z-score technique entails selecting a target chromosome, measuring the cell-free DNA (cfDNA) fragment count of the target chromosome in the pregnant woman's blood and the cfDNA fragment count of all the chromosomes in the pregnant woman's blood, and defining yₖ as the ratio of the cfDNA fragment count of the target chromosome to the cfDNA fragment count of all the chromosomes, so as to compare the subject's yₖ value and the yₖ value of a reference data set from maternal plasma of euploid pregnancies and thereby calculate the probability that the subject will develop fetal chromosomal aneuploidy.

Researchers put forth another blood screening method known as Normalized Chromosome Value (NCV) technique which entails selecting yₖ and a reference chromosome, wherein there is a positive correlation of sequencing read number between the reference chromosome and the target chromosome, and defining y_{R} as the ratio of the cfDNA fragment count of the reference chromosome to the cfDNA fragment count of all the chromosomes, so as to calculate a Sₖ value. The Sₖ value equals the ratio of yₖ to y_{R}.

For example, a medical study indicates that, among human beings, chromosome 9 is positively correlated with chromosome 21 in terms of the amount of plasma cfDNA fragment. Hence, to screen for Down's Syndrome (21-trisomy syndrome or T21), it is feasible to treat chromosome 21 as the target chromosome, and chromosome 9 as the reference chromosome.

Z-score technique and NCV technique refer to the number of standard deviations from the mean of a reference data set. Hence, when compared with the mean and standard deviation of chromosome 21 values of the reference data set, a high z-score or NCV-score for chromosome 21 indicates fetuses with T21.

In general, maternal blood screening is performed at or after 12^{th} gestational weeks because the amount of cffDNA fragments is too low to analyze in early pregnancy. The amount of cffDNA fragments in a pregnant woman's plasma increases with the gestational week; there are more cffDNA fragments in the second trimester than in the first trimester, and thus maternal blood screening performed in the second trimester is theoretically easy and reliable than it is in the first trimester since more cffDNA fragments available for analysis. However, pregnant women always hope to know as early as possible whether their fetuses have normal chromosomes.

In view of this, the inventor contemplates and studies the aforesaid issues and eventually invents a non-invasive prenatal testing method based on whole-genome tendency scoring.

### SUMMARY OF THE INVENTION

The present invention provides a non-invasive prenatal testing method, which is based on genome-wide normalized score (GWNS) to perform non-invasive prenatal screening in the first trimester and with high precision.

In order to achieve the above and other objectives, the present invention provides a non-invasive prenatal testing method, which is based on GWNS to test whether a target fetus has autosomal aneuploidy, the testing method comprising the steps of:

(a) preparing control plasma samples and a target plasma sample, wherein the control plasma samples are from pregnant women with euploid fetuses, and the target plasma sample is from a pregnant woman with the target fetus;

(b) sequencing and obtaining the amount of total cfDNA and the amount of cfDNA of target chromosome k from each of the control plasma samples and the target plasma sample, wherein the target chromosome k is selected from human chromosomes 1 to 22;

(c) respectively obtaining yₖ values from the control plasma samples and the target plasma sample, wherein each yₖ value is the ratio of the amount of cfDNA of target chromosome k to the amount of total cfDNA in each of the control plasma samples and the target plasma sample, wherein the yₖ values from the control plasma samples are denoted as control yₖ values, and the yₖ value from the target plasma sample is denoted as target yₖ value;

(d) obtaining a mₖ value, which is a mean value of the control yₖ values, wherein Σₖ₌₁²²mₖ=1;

(e) creating a reference dataset, which is collected from the ratios of each of the control yₖ values to the mₖ value;

(f) obtaining a target Rₖ value, which is a normalized ratio of the target yₖ value to the mₖ value; and

(g) comparing whether the target Rₖ value is significantly different from the reference dataset.

Preferably, the step of sequencing and obtaining the amount of total cfDNA and the amount of cfDNA of target chromosome k from each of the control plasma samples and the target plasma sample is by Next Generation Sequencing (NGS).

Preferably, the target plasma sample further comprises the maternal cfDNA and the cffDNA, wherein the concentration of the cfMNA is ≧4%.

More preferably, the target plasma sample further comprises the maternal cfDNA and the cffDNA, wherein the concentration of the cell-free fetal DNA is from 4% to 20%.

Preferably, the pregnant woman with the target fetus is at the 10^{th} to 30^{th} gestational weeks of pregnancy.

More preferably, the pregnant woman with the target fetus is at the 10^{th} to 12^{th} gestational weeks of pregnancy.

Preferably, the target chromosome k comprises a chromosome selected from the group consisting of human chromosome 13, human chromosome 18, and human chromosome 21.

According to the present invention, the advantages of the non-invasive prenatal testing method are as follows: increasing comparable data and thus enhancing the accuracy of the test, by comparing the target Rₖ value of the target pregnant woman and the values of the reference dataset; given a genome-wide normalized score (GWNS) was examined for all the human chromosomes (that is, the target chromosome k can be any one of chromosomes 1 to 22), and thus the non-invasive prenatal testing method of the present invention can be used to test aneuploidy for all human chromosomes; given the lower requirement for the concentration of cffDNA in maternal plasma, the non-invasive prenatal testing method of the present invention can be performed in earlier stage (10^{th} gestational weeks) of pregnancy with high accuracy, such that the pregnant woman can know as early as possible whether her fetus has normal chromosomes; and the non-invasive prenatal testing method of the present invention merely requires sampling the pregnant woman's blood sample and thus is a non-invasive medical procedure which is safe and reliable to the fetus and the pregnant woman.

Other objectives, advantages and novel features of the invention will become more apparent from the following detailed description when taken in conjunction with the accompanying drawings.

### BRIEF DESCRIPTION OF THE DRAWINGS

Objectives, features, and advantages of the present invention are hereunder illustrated with specific embodiments in conjunction with the accompanying drawings, in which:

FIG. 1 is a flow chart of an embodiment of the present invention;

FIG. 2 is a schematic view of the result of example 1 performed according to the present invention, wherein the percentages are the concentrations of cffDNA of each of the target samples;

FIG. 3 is a schematic view of simulation data of comparison of the present invention and the other two conventional methods;

FIG. 4 (PRIOR ART) is a schematic view of the result of a clinical experiment performed by conventional Z-score method, wherein the percentages are the concentrations of cffDNA of each of the target samples; and

FIG. 5 (PRIOR ART) is a schematic view of the result of a clinical experiment performed by conventional NCV method, wherein the percentages are the concentrations of cffDNA of each of the target samples.

### DETAILED DESCRIPTION OF THE PREFERRED EMBODIMENTS

Like components described hereunder are denoted with like reference numerals.

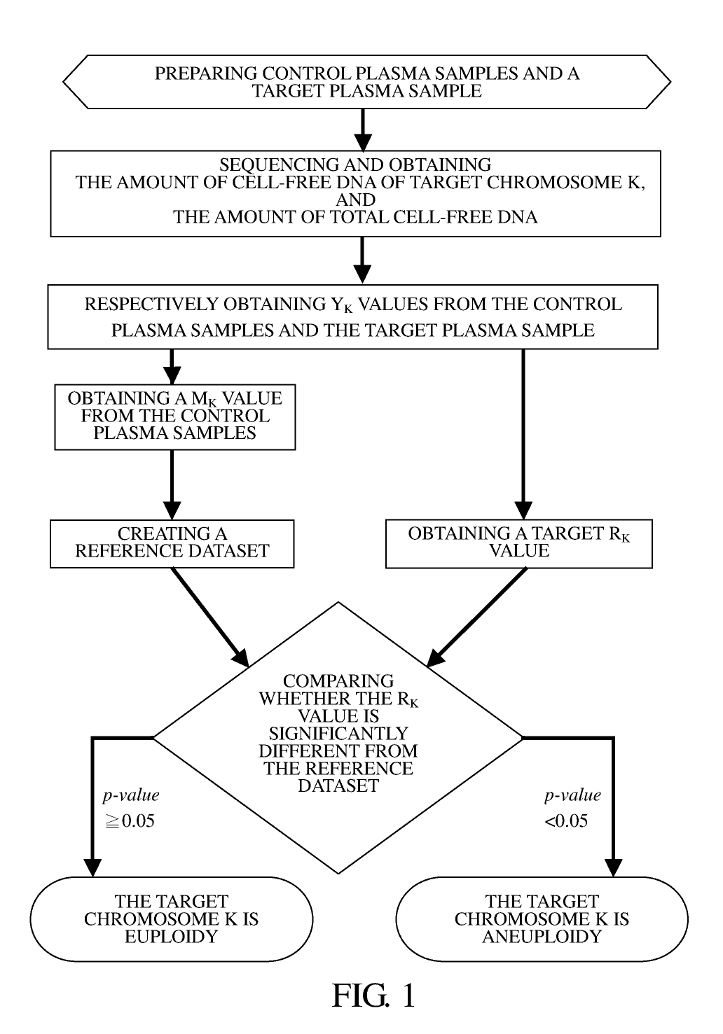

Referring to FIG. 1, there is shown a diagram of a non-invasive prenatal testing method, which is based on genome-wide normalized score (GWNS) to test whether a target fetus has autosomal aneuploidy, the testing method comprising the steps of:

preparing control plasma samples and a target plasma sample, wherein the control plasma samples are from pregnant women with euploid fetuses, and the target plasma sample is from a pregnant woman with the target fetus;

sequencing and obtaining the amount of total cfDNA and the amount of cfDNA of target chromosome k from each of the control plasma samples and the target plasma sample, wherein the target chromosome k is selected from human chromosomes 1 to 22;

respectively obtaining yₖ values from the control plasma samples and the target plasma sample, wherein each yₖ value is the ratio of the amount of cfDNA of the target chromosome k to the amount of total cfDNA in each of the control plasma samples and the target plasma sample, wherein the yₖ values from the control plasma samples are denoted as control yₖ values, and the yₖ value from the target plasma sample is denoted as target yₖ value;

obtaining a mₖ value, which is a mean value of the control yₖ values, wherein Σₖ₌₁²²mₖ=1;
creating a reference dataset, which is collected from the ratios of each of the control yₖ values to the mₖ value;
obtaining a target Rₖ value, which is a normalized ratio of the target yₖ value to the mₖ value; and
comparing whether the target Rₖ value is significantly different from the reference dataset.
If the Rₖ value is significantly different from the reference dataset (p-value<0.05), the target chromosome k is aneuploidy, if not, the target chromosome k is euploidy (p-value≧0.05).

### Preparation 1 Blood Collection and DNA Extraction

Peripheral blood samples were collected from pregnant women at 10 weeks of gestation. Each of the plasma samples was obtained from each peripheral blood sample by double centrifugations with 1,600×g for 10 minutes at 4° C., following by centrifugation at 16,000×g for 10 minutes at 4° C. Each of the plasma samples was divided into 1 mL aliquots in separate eppendorf tubes and stored at −80° C. until subsequent analysis.

CfDNA was extracted from 1 mL of the plasma sample and eluted in 30 μL of DNase-free H₂O with QlAamp Blood Mini Kit (Qiagen, Hilden, Germany) according to the blood and body fluid protocol. Genomic DNA from maternal blood cells was also extracted by using WelPrep DNA kit (Welgene Biotech, Taipei, Taiwan) according to the manufacturer's instructions. DNA concentration was examined with Qubit Fluorometer (Invitrogen, Calif., USA).

This preparation is both applicable to the control plasma samples and the target plasma samples.

### Preparation 2 DNA Sequencing by Next Generation Sequencing (NGS)

After the steps of preparation 1, the total cfDNA read number, the target chromosome k cfDNA read number, and the cffDNA concentration of each plasma sample were counted by massively parallel sequencing.

This preparation is both applicable to the control plasma samples and the target plasma samples.

### Preparation 3 Calculation of yₖ Value

To each sample, the ratio of the target chromosome k cfDNA read number to the total cfDNA read number was denoted as a yₖ value. The yₖ values from the control plasma samples were denoted as control yₖ values, and the yₖ value from the target plasma sample was denoted as target yₖ value.

### Preparation 4 Creation of a Reference Dataset

The reference dataset is collected from the ratios of each of the control yₖ values to the mean value from those control yₖ values (mₖ value) from preparation 3.

In this preparation, there are 55 ratios to the reference dataset.

### EXAMPLE 1

### Detection Trisomy 21 (T21) of the Target Fetus by GWNS, Z-Score, and NCV Testing Methods

In this example, the target chromosome k is chromosome 21 (k=21), 128 disomy 21 (D21), including 124 euploid and four trisomyl8 (T18) cases, and 25 T21 pregnancies were used as the test sample set. Furthermore, in order to confirm the sensitivity of the three different testing methods, the plasma samples of four T21 pregnant women were serially diluted by maternal blood DNA thereof, wherein the fetal DNA concentration varied from 1.59% to 18.29%.

A target y₂₁ value (target yₖ value, k=21) of each of the target samples above was obtained by the steps of preparations 1 to 4. Then each of the target R₂₁ values, which were the ratio of each of the target y₂₁ values to the mean value from those control y₂₁ values of the database in preparation 4, was obtained. Comparing each target R₂₁ value to the reference dataset in preparation 4, if the target R₂₁ value is significantly different from the reference dataset (p-value<0.05), the target sample has chromosome 21 aneuploidy.

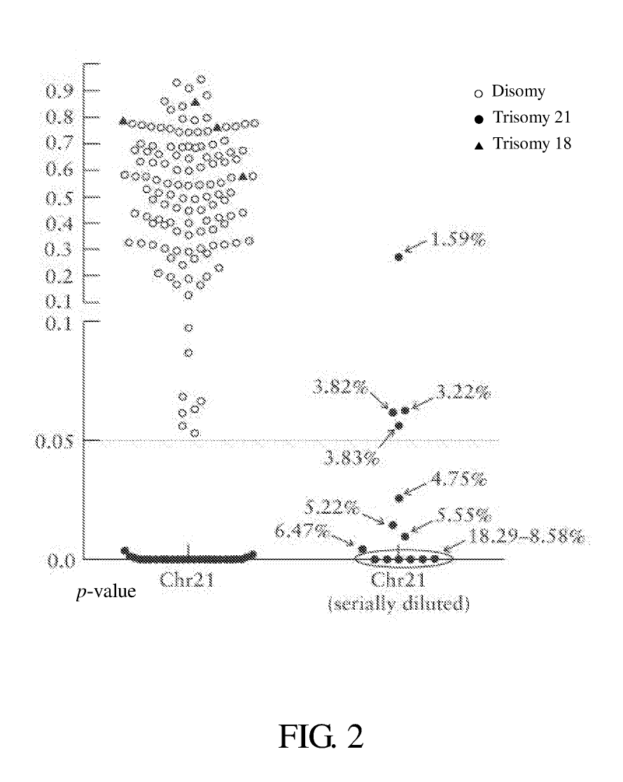

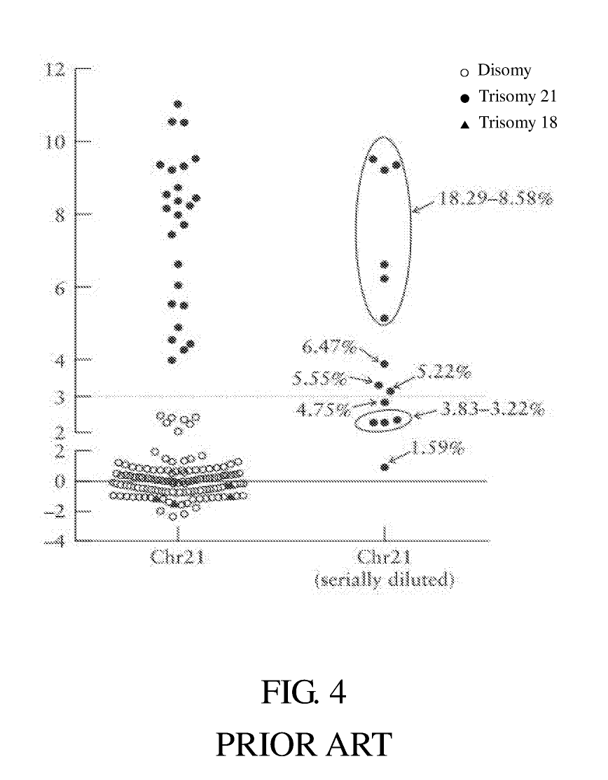

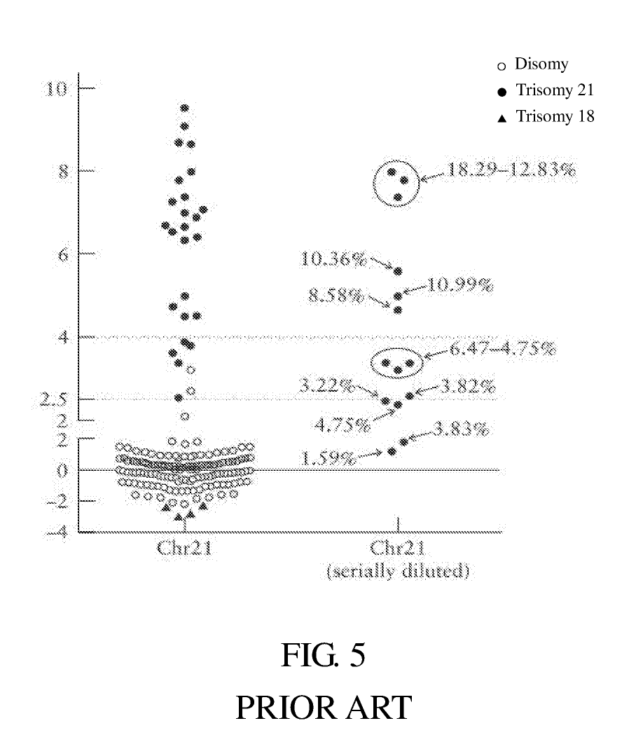

Referring to FIGS. 2, 4 and 5, all of the 25 T21 pregnant women, whose fetuses were diagnosed with Down's syndrome (T21), could be detected by the method of the present invention shown in FIG. 2 and Z-score method shown in FIG. 4. The results were comparable to those of the method of the present invention and Z-score, and were superior to those of NCV for which four samples with values of 2.5<NCV<4.0 were classified as ‘no call’, as shown in FIG. 5.

Referring to FIGS. 2, 4 and 5, the serially diluted T21 samples were detected in cffDNA concentration 4.75% to 18.29% by the method of the present invention, whereas, the minimums of the cffDNA that could be detected were 5.22% by Z-score and 8.58% by NCV, respectively. These results indicated that the method of the present invention can accurately detect T21 in lower cffDNA concentration than Z-score and NCV, and thus can be performed in earlier stage during the pregnancy.

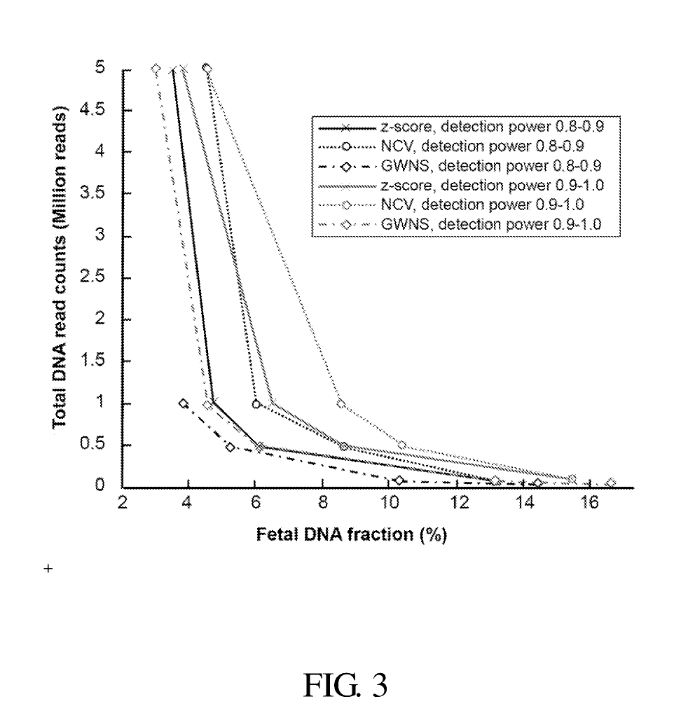

Referring to FIG. 3, in order to understand whether the method of the present invention is effective in performing T21 screening or other chromosomal quantity abnormality screening during the early pregnancy, the inventor of the present invention performs iso-quality curve comparison on NGS sequencing results obtained from the samples of 64 pregnant women with fetuses free of chromosomal quantity abnormality and 22 pregnant women with fetuses diagnosed with T21 abnormality. Then, the inventor analyzes the relation between the NGS DNA sequencing quantity required for Z-score method, NCV method, and the method of the present invention and the cffDNA concentration required for Z-score method, NCV method, and GWNS, under a specific test accuracy, and then plots a broken line graph shown in FIG. 3. As shown in FIG. 3, the horizontal axis represents the concentration of cffDNA in the sample, wherein a low concentration of cffDNA in the sample indicates a pregnant woman's sample obtained from earlier stage of pregnancy. By contrast, a high concentration of cffDNA in the sample indicates a pregnant woman's sample that may obtain in late stage of pregnancy. The vertical axis represents the NGS DNA sequencing quantity which has to be read (that is, the NGS read number, also the total DNA read counts) so as for a technique/method to be performed to achieve specific test accuracy at a specific cffDNA concentration fraction. As shown in the diagram, the method of the present invention always features a number line lower than that of the other two conventional methods, under a specific test accuracy. Hence, to reach the same test accuracy, the method of the present invention requires lower NGS read number for a given cffDNA concentration or required lower cffDNA concentration for a given NGS read number than the other two conventional methods in T21 screening or the other chromosomal aneuploidy screening. According to FIG. 2, the method of the present invention can accurately detect T21 in lower cffDNA concentration (more than 3.8%); therefore, the total read counts only need 1 million reads in FIG. 3.

### EXAMPLE 2

### Detection of Other Chromosome Aneuploidy (T13, T18) as Positive Control by the Non-Invasive Prenatal Testing Method of the Present Invention

In order to confirm the non-invasive prenatal testing method of the present invention could also detect the other chromosome, in addition to chromosome 21, aneuploidy, in this example, there were 81 fetuses diagnosed with trisomy 13, trisomy 18, or trisomy 21. All of the 81 target samples could be detected by the non-invasive prenatal testing method of the present invention according to the foregoing preparations 1 to 4, and results were confirmed by invasive tests with fluorescence in situ hybridization (FISH) or cytogenetic analysis. The result of example 2 is shown in Table 1 as follows:

TABLE 1
Detection of T13, T18, and T21 by the non-invasive
prenatal testing method of the present invention

| | Method | FISH or cytogenetic analysis | | | |
|---|---|---|---|---|---|
| Method | Trisomy | T13 | T18 | T21 | Total |
| the present | T13 | 13 | 0 | 0 | 13 |
| invention | T18 | 0 | 44 | 0 | 44 |
| | T21 | 0 | 0 | 24 | 24 |

| | | | | |
|---|---|---|---|---|
| Total | 13 | 44 | 24 | 81 |

As shown in Table 1, when the target chromosome k is human chromosome 13, 13 target fetuses were correctly detected with human chromosome 13 aneuploidy, when the target chromosome k is human chromosome 18, 44 target fetuses were correctly detected with human chromosome 18 aneuploidy, and when the target chromosome k is human chromosome 21, 24 target fetuses were correctly detected with human chromosome 21 aneuploidy. The results were consistent with that of FISH or cytogenetic analysis. Therefore, the non-invasive prenatal testing method of the present invention could correctly detect the trisomy 13, trisomy 18, and trisomy 21.

### EXAMPLE 3

### Clinical Test

Generally, fetuses could still survive despite they have trisomy 13, trisomy 18, or trisomy 21. Hence, in the clinical test, 1022 pregnant women were as target samples.

The target samples were detected by the non-invasive prenatal testing method of the present invention according to the foregoing preparations 1 to 4, and subsequently confirmed by invasive tests with FISH or cytogenetic analysis. The result of example 3 is shown in Table 2 as follows:

TABLE 2
Clinical test of 1022 pregnant women by the non-invasive
prenatal testing method of the present invention

| | | FISH or cytogenetic analysis | | | | |
|---|---|---|---|---|---|---|
| Method | Method | T13 | T18 | T21 | Normal | Total |
| the present | T13 | 2 | 0 | 0 | 0 | 2 |
| invention | T18 | 0 | 1 | 0 | 0 | 1 |
| | T21 | 0 | 0 | 6 | 2 | 8 |
| | Normal | 0 | 0 | 0 | 1011 | 1011 |

| | | | | | |
|---|---|---|---|---|---|
| Total | 2 | 1 | 6 | 1013 | 1022 |

As shown in Table 2, for trisomy 13 (T13) and trisomy 18 (T18), the sensitivity and specificity of the method of the present invention were both 100%. For trisomy 21 (T21), there were 8 target samples classified as T21 by the method of the present invention, though 2 of the 8 T21 were confirmed as normal fetuses by FISH or cytogenetic analysis (that is, there are 2 false positive cases). Therefore, for T21, the sensitivity and specificity of the method of the present invention were 100% and 99.8%, respectively.

As indicated above, the present invention has industrial applicability, novelty, and non-obviousness.

What is claimed is:

1. A non-invasive prenatal testing method, which is based on genome-wide normalize score (GWNS) to test whether a target fetus has autosomal aneuploidy, the testing method comprising the steps of:
(a) preparing control plasma samples and a target plasma sample, wherein the control plasma samples are from pregnant women with euploid fetuses, and the target plasma sample is from a pregnant woman with the target fetus;
(b) sequencing and obtaining the amount of total cfDNA and the amount of cfDNA of target chromosome k from each of the control plasma samples and the target plasma sample, wherein the target chromosome k is selected from human chromosomes 1 to 22;
(c) respectively obtaining yₖ values from the control plasma samples and the target plasma sample, wherein each yₖ value is the ratio of the amount of cfDNA of target chromosome k to the amount of total cfDNA in each of the control plasma samples and the target plasma sample, wherein the yₖ values from the control plasma samples are denoted as control yₖ values, and the yₖ value from the target plasma sample is denoted as target yₖ value;
(d) obtaining a mₖ value, which is a mean value of the control yₖ values, wherein Σₖ₌₁²²mₖ=1;
characterized in:
(e) creating a reference dataset, which is collected from the ratios of each of the control yₖ values to the mₖ value;
(f) obtaining a target Rₖ value, which is a normalized ratio of the target yₖ value to the mₖ value; and
(g) comparing whether the target Rₖ value is significantly different from the reference dataset.

2. The non-invasive prenatal testing method according to claim 1, wherein the step of obtaining the amount of cfDNA of target chromosome k and the amount of total cfDNA from each of the plasma samples is by Next Generation Sequencing (NGS).

3. The non-invasive prenatal testing method according to claim 1, wherein the target plasma sample further comprises the maternal cfDNA and the cffDNA, wherein the concentration of the cffDNA is 4%.

4. The non-invasive prenatal testing method according to claim 3, wherein the target plasma sample further comprises the maternal cfDNA and the cffDNA, wherein the concentration of the cffDNA is from 4% to 20%.

5. The non-invasive prenatal testing method according to claim 1, wherein the pregnant woman with the target fetus is at the 10^{th} to 30^{th} gestational weeks of pregnancy.

6. The non-invasive prenatal testing method according to claim 5, wherein the pregnant woman with the target fetus is at the 10^{th} to 12^{th} gestational weeks of pregnancy.

7. The non-invasive prenatal testing method according to claim 1, wherein the target chromosome k comprises one selected from the group consisting of human chromosome 13, human chromosome 18, and human chromosome 21.
